(19) United States
(12) Patent Application Publication
(10) Pub. No.: US 2018/0192945 A1
(43) Pub. Date: Jul. 12, 2018

(54) METHODS FOR PREDICTING COMPOSITIONAL BODY CHANGES

(71) Applicant: KLARISMO, INC., San Francisco, CA (US)

(72) Inventors: Ignatius Dewet Diener, London (GB); Marcus Foster, San Francisco, CA (US); David Greer, London (GB); Kevin Keraudren, London (GB); Brandon Whitcher, London (GB)

(21) Appl. No.: 15/419,998

(22) Filed: Jan. 30, 2017

Related U.S. Application Data
(63) Continuation of application No. PCT/US17/12891, filed on Jan. 10, 2017.
(60) Provisional application No. 62/450,021, filed on Jan. 24, 2017.

(51) Int. Cl. A61B 5/00; G06T 7/62; G06T 7/00; G06T 7/10; G06T 7/33; A61B 5/055; A61B 6/03

(52) U.S. Cl. CPC A61B 5/4872; G06T 7/62; G06T 7/0016; G06T 7/10; G06T 7/33; G06T 2207/30204; A61B 6/032; G06T 2207/10088; G06T 2207/30004; G06T 2207/30196; G06T 2207/20076; A61B 5/055

(57) ABSTRACT

Within this disclosure are new methods of predicting compositional changes of a body. In one embodiment, the methods disclosed herein comprise three-dimensional models of a body. In one embodiment, MRI data is used to create a three-dimensional model of a body. In one embodiment, a stimulus is chosen to influence change in a body. In one embodiment, comparator bodies are used to predict changes to the body.

### CROSS REFERENCE TO RELATED APPLICATIONS

This application is a Continuation of International Application Serial Number PCT/US17/12891, entitled “METHODS FOR PROCESSING THREE DIMENSIONAL BODY IMAGES,” filed Jan. 10, 2017 and claims benefit of Provisional Application Ser. No. 62/450,021, entitled “METHODS FOR PREDICTING COMPOSITIONAL BODY CHANGES,” filed Jan. 24, 2017, which are hereby incorporated by reference in their entirety.

### TECHNICAL FIELD

This disclosure relates to body imaging and analysis. In particular, this disclosure relates to predicting changes of a body.

### BACKGROUND

Many popular trends have arisen over the years promising to lower body weight and improve health. Machines, exercises, foods and supplements, etc., all promise a uniform solution to people of different ages, body types, and overall health. Yet, none of these methods focus on personal needs and wants of individuals.

Some solutions to these problems are personalized meal and exercise plans. These methods begin with an initial evaluation of the body, e.g., beginning body weight, fat to muscle ratio, current diet, level of exercise, etc. However, these methods fail to truly comprehend how one's body is composed.

To that end, internal imaging is one solution. Internal imaging provides an in-depth look at the body and allows one to create a truly personalized health plan suited to his or her needs. Common medical imaging techniques include X-ray, Magnetic Resonance Imaging (MRI), and Computerized Tomography (CT) scans. One advantage MRI scans have over CT scans is that MRI does not expose the patient to ionizing radiation that could cause potential side effects. MRI scans provide a high level of detail, resolution, and clarity of the anatomy and physiology of the human body.

The downfall of these methods however is the lack of usability. An MRI scan collects an extraordinary amount of information about the human body but much of the information is left unused. Only certain medical professionals are provided with the training and equipment to fully extrapolate all the information from an MRI scan. While a patient may receive the actual MRI data, the patient is left without the proper tools to fully utilize the data for their own benefit. Also, the current level of technology does not allow for one to easily manipulate data for predicting how certain changes will affect the body. Another limitation of the current state of the art is that radiologists perform qualitative assessments of body images whereas analyzing the images quantitatively would provide for improvements in speed, efficiency, accuracy, etc.

There exists a need for predicting compositional changes to a body. There also exists a need for using MRI scans to predict changes to a body. There exists a need for easy and efficient use of MRI data. There also exists a need for predicting changes by using various stimulus. There exists a need for calculating and predicting volumetric measurements of a body.

### DETAILED DESCRIPTION

Disclosed herein is a new method of predicting compositional changes to a body. In one embodiment, predicting compositional changes comprises utilizing a three-dimensional representation of the body. In one embodiment, the body is landmarked and segmented for predicting future changes. In one embodiment, there is a plurality of three-dimensional representations. In one embodiment, a stimulus is applied to a first segment. In one embodiment, the stimulus is exercise. In one embodiment, the stimulus is diet. In one embodiment, predicting compositional changes to a body comprises utilizing a machine learning program. In one embodiment, predicting compositional changes to a body comprises a cloud database platform.

Disclosed herein is a new method of calculating volumetric measurements of a body. In one embodiment, the volume of a first segment from a first body is calculated at a first age. In one embodiment, the volume of a second segment from a first body is calculated from at a second age. In one embodiment, the volume of a plurality of segments from a plurality of bodies is measured at a plurality of ages.

Disclosed herein is a new method of internal imaging. In one embodiment, MRI images are used to construct three-dimensional representations of a body to predict changes. In one embodiment, there is a plurality of MRI images from a plurality of bodies from a plurality of ages.

Disclosed herein is a method of predicting compositional change in a body comprising:
Collecting a plurality of two-dimensional images for a first body;
Collecting a plurality of two-dimensional images for a second body;
Processing the plurality of two-dimensional body images for the first body into a first three-dimensional representation of the first body;
Processing the plurality of two-dimensional body images for the second body into a second three-dimensional representation of the second body;
Assigning landmarks to points within the first three-dimensional representation of the first body;
Assigning landmarks to points within the second three-dimensional representation of the second body;
Registering the first three-dimensional representation of the first body;
Registering the second three-dimensional representation of the second body;
Segmenting the first three-dimensional representation of the first body into a first segment;
Segmenting the second three-dimensional representation of the second body into a second segment;
Calculating a volume of the first segment;
Calculating a volume of the second segment;
Identifying a first stimulus; and
Predicting a change in the volume of the first segment.

As used herein, the term “collecting” means gathering, receiving, transferring, downloading, and/or finding. In one embodiment, collecting comprises receiving information from a cloud database. In one embodiment, collecting comprises transferring information from a disk onto a computer. In one embodiment, collecting comprises transferring information from a USB drive onto a computer. In one embodiment, collecting comprises streaming. In one embodiment, collecting is gathering data for calculating differences. In one embodiment, calculating comprises collecting information for predicting changes.

In one embodiment, collecting comprises receiving images from an MRI machine. An MRI machine changes the orientation of protons in the body using magnetic energy. As the protons change to their original orientation, they produce radio signals which are recorded. Protons in different tissues of the body behave differently, producing different radio signals allowing an MRI machine to differentiate the different tissues of the body. The intensity of the received signal is then plotted on a grey scale and cross sectional images are built. In one embodiment, the MRI machine stores images in DICOM format. In one embodiment, collecting comprises receiving multiple images from multiple MRI machines. In one embodiment, collecting comprises receiving multiple images from multiple MRI machines from multiple points in time. In one embodiment, MRI scans are collected from a plurality of bodies.

Within the context of this disclosure, collecting encompasses passively receiving information in which information is collected without request. Within the context of this disclosure, collecting encompasses actively receiving information in which information is requested.

As used herein, the term “plurality” refers to more than one. In one embodiment, there is a plurality of images for one body. In one embodiment, there is a plurality of two-dimensional images of the body. In one embodiment, there is a plurality of planar slices of the body. In one embodiment, there is a plurality of parts of the same body. In one embodiment, there is a plurality of images for a plurality of bodies. In one embodiment, there is a plurality of images for one section of a body. In one embodiment, there is a plurality of bodies. In one embodiment, there is a plurality of changes. In one embodiment, there is a plurality of predictions.

As used herein, the term “two-dimensional” refers to an object or image appearing to have width and length, i.e., area, within a plane. Area is the quantity that expresses the extent of a two-dimensional figure or shape in the plane. In one embodiment, two-dimensional comprises Cartesian coordinates. In one embodiment, an image is in the x and y axis. In one embodiment, a body is in the x and z axis. In one embodiment, a body is in the y and z axis. In one embodiment, two dimensional comprises polar coordinates.

As used herein, the term “image” refers to a likeness or representation of a person, animal, or thing, photographed, painted, sculptured, and/or otherwise made visible. In one embodiment, an image is a digital image. In one embodiment, an image is a picture of a human body. In one embodiment, an image is from an MRI machine. In one embodiment, an image is a collection of pixels. In one embodiment, the image is in a JPEG format. In one embodiment, the image is in DICOM format. In one embodiment, the image is in NIfTI format. In one embodiment, the image illustrates a change in a body.

As used herein, the term “two-dimensional image” refers to a presentation of mass present within a particular plane, e.g., cross-section of a body. In one embodiment, the two-dimensional image comprises Cartesian coordinates, e.g., x, y, and z axis. In one embodiment, the two-dimensional image is a picture. In one embodiment, the two-dimensional image is a digital image on a screen. In one embodiment, there are two-dimensional images from two time periods.

As used herein, the term “body” refers to the physical structure and the material substance of a mass, e.g., an organism. In one embodiment, the body is living. In one embodiment, the body is not living. In one embodiment, the body is a human. In one embodiment, the body is a dog. In one embodiment, the body is a horse. In one embodiment, the body comprises organs. In one embodiment, the body comprises a skeleton. In one embodiment, the body comprises fat. In one embodiment, the body comprises muscles. In one embodiment, the body comprises subcutaneous fat. In one embodiment, the body comprises adipose tissue. In one embodiment, the body comprises visceral fat. In one embodiment, the body comprises iliopsoas muscles.

As used herein, the term “image of a body” refers to a representation of a physical structure and material substance of a physical mass, e.g., an organism. In one embodiment, the image of a body is an embodiment of an MRI scan. In one embodiment, the image of a body is an embodiment of a CT scan. In one embodiment, the image of a body is an internal image. In one embodiment, the image of a body is of a human body or a portion of a human body. In one embodiment, there is an image of a first body. In one embodiment, there is an image of a second body. In one embodiment, there is an image of a third body. In one embodiment, there is an image of a plurality of bodies.

As used herein, the term “processing” refers to treating a thing through a series of steps. In one embodiment, processing comprises using a computer algorithm. In one embodiment, processing comprises producing an image from another image. In one embodiment, processing comprises using a processor. In one embodiment, processing comprises manually manipulating an image. In one embodiment, processing comprises creating a three-dimensional image from a series of two-dimensional crosscut images. In one embodiment, processing comprises analyzing segments of a body. In one embodiment, processing comprises predicting based on previously collected data.

As used herein, the term “three-dimensional” refers to an object or image appearing to have length, depth, and breadth, i.e., volume. Volume is the amount of space occupied by an object. In one embodiment, volume is measured in cubic meters (m3). In one embodiment, three dimensional comprises Cartesian coordinates. In one embodiment, an image has multiple x, y, and z coordinates.

As used herein, the term “representation of the body” refers to a concrete portrayal of a physical structure having material substance. In one embodiment, the representation of the body is two-dimensional. In one embodiment, the representation of the body is three-dimensional. In one embodiment, the representation of the body is a representation of a human body. In one embodiment, the representation of the body is a picture. In one embodiment, the representation of the body is a digital image. In one embodiment, the representation of the body is a representation of the skeleton. In one embodiment, the representation of the body is an MRI scan. In one embodiment, the representation of the body is a CT scan. In one embodiment, the representation of the body is an X-ray. In one embodiment, a representation of the body shows changes. In one embodiment, a representation of a body is a prediction of future changes.

As used herein, the term “assigning” refers to categorizing or labeling specific things, e.g., points, areas, or volumes of the body. In one embodiment, assigning refers to marking areas of the body with a machine learning program. In one embodiment, assigning refers to detecting landmarks. In one embodiment, assigning comprises marking landmarks to make predictions. In one embodiment, assigning comprises labeling future changes.

As used herein, the term “landmark” refers to a particular reference part of a thing. In one example, an area of the body where a bone is on the surface. Landmarks are often used as a reference point of the body to find other structures. In one embodiment, a landmark is used to measure proportion. In one embodiment, a landmark is used to find a form. In one embodiment, a landmark is a bone joint. In one embodiment, a landmark is the vertebrae. In one embodiment, a landmark is the armpit. In one embodiment, a landmark is a spine curve. In one embodiment, a landmark is a shoulder; left, right, or both. In one embodiment, a landmark is a hip; left, right, or both. In one embodiment, a landmark is a knee; left, right, or both. In one embodiment, a landmark is an ankle; left, right, or both. In one embodiment, the landmarks are based on a coordinate system unique to an individual body. In one embodiment, a landmark is used as a reference point for monitoring changes of a body.

As used herein, the term “assigning landmark” refers to designating, labeling, and/or marking a particular reference part of a thing. In one embodiment, assigning a landmark comprises labeling a bone joint. In one embodiment, assigning a landmark comprises labeling the centerline of the front of the body. In one embodiment, assigning a landmark comprises labeling the centerline of the back. In one embodiment, assigning a landmark comprises labeling the shoulder; left, right, or both. In one embodiment, assigning a landmark comprises labeling the hip; left, right, or both. In one embodiment, assigning a landmark comprises labeling the knee; left, right, or both. In one embodiment, assigning a landmark comprises labeling the ankle; left, right, or both. In one embodiment, assigning a landmark is for predicting changes around the landmark. In one embodiment, multiple landmarks are assigned to predict multiple changes.

As used herein, the term “point within the three-dimensional representation of the body” refers to a specific position within a portrayal of a physical structure with reference to the physical structure's length, width, and height/depth. In one embodiment, the point within the three-dimensional representation of the body is a landmark. In one embodiment, the point within the three-dimensional representation of the body is a bone joint. In one embodiment, the point within the three-dimensional representation of the body is a body part. In one embodiment, the point within the three-dimensional representation of the body is the chest. In one embodiment, a point within the three-dimensional representation of the body is for predicting changes occurring around that position.

As used herein, the term “registering” refers to matching and/or aligning two objects based on a set of features. In one embodiment, “registering” means aligning two images, such as landmarks of a representation of a body. In one example, registering means finding the warping transforming an image so that it best fits another image. In one embodiment, registering means finding a transformation mapping an image onto another image. In one embodiment, registering allows subsequent processes to recognize an image quicker and more efficiently. In one embodiment, registering comprises recording an image of a specific landmark common to multiple bodies. In one embodiment, registering comprises utilizing a cloud database platform. In one embodiment, registering comprises implementing non-rigid registration. In one embodiment, non-rigid registration is elastic deformation.

As used herein, the term “segmenting” refers to dividing, cutting, isolating, and/or segregating a thing into separate pieces or sections. Segmenting allows one to focus on an area of interest and study that area in more detail. In one embodiment, segmenting comprises dividing pieces of a body. In one embodiment, segmenting comprises dividing images of a body. In one embodiment, segmenting comprises dividing a body into a left and right half. In one embodiment, segmenting comprises dividing the body into the head, torso, left leg, and right leg. In one embodiment, segmenting comprises dividing the torso into the chest, back, abdomen, and shoulders. In one embodiment, segmenting comprises dividing the leg muscles into the upper and lower segments. In one embodiment, segmenting comprises dividing the leg muscles into left and right segments. In one embodiment, segmenting comprises dividing the leg bones into the femur and tibia. In one embodiment, segmenting comprises using atlas based segmentation of the kidneys. In one embodiment, segmenting comprises using atlas based segmentation of the liver. In one embodiment, segmenting does not separate the segments from the whole. In one embodiment, segmenting is for focusing on particular areas of change. In one embodiment, segmenting comprises predicting changes to particular segments.

As used herein, the term “segment” refers to an individual section or piece of a thing. In one embodiment, the segment is attached to the thing. In one embodiment, the segment is separated from the thing. In one embodiment, a body is separated. In one embodiment, a segment of the body is an arm. In one embodiment, a segment of the body is the torso. In one embodiment, a segment of the body is separated into more segments. In one embodiment, an arm is segmented into fingers, hand, elbow, shoulder, etc. In one embodiment, a segment is a tissue of the body. In one embodiment, a segment is used to predict changes between two bodies. In one embodiment, a segment is used as a reference point to predict changes compared to other segments.

As used herein, the term “calculating” refers to determining a value through a computation or computations. In one embodiment, calculating comprises using a machine learning program. In one embodiment, calculating comprises manual computations. In one embodiment, calculating comprises determining the volume of a segment. In one embodiment, calculating comprises determining the amount of fat in a torso. In one embodiment, calculating comprises measuring the muscle mass of an arm. In one embodiment, calculating comprises determining the volume difference between two segments. In one embodiment, calculating comprises measuring the fat difference between representations of a body. In one embodiment, calculating comprises predicting value changes over time. In one embodiment, calculating comprises determining the accuracy of predictions. In one embodiment, calculating comprises developing new predictions based on new information.

As used herein, the term “machine learning program” refers to a type of artificial intelligence providing an apparatus, e.g., a computer, the ability to comprehend material without being explicitly instructed. In one embodiment, a machine learning program is taught to learn languages. In one embodiment, a machine learning program predicts the weather based on weather changes. In one embodiment, a machine learning program filters spam. In one embodiment, a machine learning program is taught to mark landmarks in different bodies. In one embodiment, the machine learning program is taught to detect centroids of the vertebrae (e.g., L5-T8). In one embodiment, a machine learning program is taught to predict changes to a body over time.

As used herein, the term “volume” refers to the amount of three-dimensional space. Examples of measurements of volume include, but are not limited to, cubic inches, cubic feet, cubic centimeters, cubic meters, cubic millimeters, and cubic liters. In one embodiment, volume correlates with the distribution of weight within the body. In one embodiment, volume comprises the fat content of the body. In one embodiment, volume comprises a measurement of total body fat. In one embodiment, volume comprises a measurement of total muscle tissue. In one embodiment, volume comprises a measurement of a muscle group. In one embodiment, volume comprises a measurement of the thigh muscle. In one embodiment, volume comprises a measurement of subcutaneous fat. In one embodiment, volume comprises a measurement of the visceral fat. In one embodiment, volume comprises a measurement of the liver fat. In one embodiment, volume comprises a measurement of the intramuscular fat. In one embodiment, the volume comprises a change in measurement.

As used herein, the term “volume of the first segment” refers to the amount of space occupied by one section or piece of a thing, e.g., those corresponding with an individual piece of the body. In one embodiment, the volume of the first segment is the weight of the body, a part of the body, or parts of the body. In one embodiment, the volume of the first segment is the total fat content of the torso. Within the context of this disclosure, similar meaning is applied to “volume of the second segment”, “volume of the third segment”, “volume of the fourth segment”, and so on. In one embodiment, the volume of the first segment is different from the volume of the second segment. In one embodiment, the volume of the first segment is predicted to match the volume of the second segment.

As used herein, the term “identifying” refers to establishing, indicating, selecting, and/or specifying. In one embodiment, identifying comprises selecting by a human operator. In one embodiment, identifying comprises a machine automatically establishing parameters. In one embodiment, identifying comprises specifying conditions.

As used herein, the term “stimulus” refers to an event, action, and/or occurrence or a series of events, actions, and/or occurrences. In one embodiment, a body reacts to a stimulus. In one embodiment, a body is predicted to react to a stimulus. In one embodiment, a stimulus caused a body to change. In one embodiment, a stimulus is predicted to change a body. In one embodiment, a stimulus comprises exercise. In one embodiment, a stimulus comprises a change in diet.

As used herein, the term “identifying a first stimulus” refers to establishing, indicating, selecting, and/or specifying an event, action, and/or occurrence or a series of events, actions, and/or occurrences. In one embodiment, identifying a first stimulus comprises selecting exercise. In one embodiment, identifying a first stimulus comprises predicting a change because of the stimulus.

As used herein, the term “predicting” means determining whether a future event will occur and/or how it will occur. In one embodiment, predicting is determining whether a three-dimensional representation of the body is accurate. In one embodiment, predicting is determining the location of segments of the body. In one embodiment, predicting refers to determining a future value when the said future value is unknown, for example, by using exogenous and/or endogenous information.

As used herein, the term “change in the volume” refers to a difference in amount of three-dimensional space. In one embodiment, a change in the volume comprises an increase in muscle volume. In one embodiment, a change in the volume comprises a decrease in the fat volume.

In one embodiment of the methods disclosed herein, predicting the change in volume comprises correlating the first stimulus with the second segment.

As used herein, the term “correlating” refers to establishing, relating, and/or connecting a relationship between two or more things in which one thing affects or depends on another. In one embodiment, correlating comprises relating a stimulus to the fat volume of a body. In one embodiment, correlating comprises establishing a relationship between a stimulus and a change in the body. In one embodiment, correlating comprises predicting a relationship between a stimulus and a change in the body. In one embodiment, correlating comprises a human operating connecting a stimulus to a change in the body. In one embodiment, correlating comprises a machine learning program establishing a relationship between a stimulus and a change in the body.

As used herein, the term “correlating the first stimulus with the second segment” refers to establishing, relating, and/or connecting a relationship between the first stimulus and second segment. In one embodiment, a first stimulus is predicted to cause a first segment to match a second segment.

In one embodiment, the methods disclosed herein comprise a plurality of second bodies.

As used herein, the term “plurality of second bodies” refers to more than one physical structures having material substance. In one embodiment, a plurality of second bodies comprises a collection of bodies having similar volumetric measurements. In one embodiment, a plurality of second bodies is a comparator. In one embodiment, the plurality of second bodies are different from the first body.

In one embodiment of the methods disclosed herein, the first stimulus is chosen from diet and exercise.

As used herein, the term “diet” refers to the food consumed by an organism, e.g., a human. In one embodiment, a diet refers to a specific regimen of food consumed by a human. In one embodiment, a diet comprises a specific regimen of food for causing a change in the body. In one embodiment, a diet is predicted to change a body.

As used herein, the term “exercise” refers to physical activity. In one embodiment, exercise is predicted to change a body. In one embodiment, exercise is any activity enhancing or maintaining physical fitness, health, and wellness.

Exercise may be performed for various reasons including, but not limited to, increasing growth and development, preventing aging, strengthening muscles and cardiovascular system, honing athletic skills, losing weight, and/or maintaining health.

In one embodiment of the methods disclosed herein, diet comprises caloric intake.

As used herein, the term “caloric intake” refers to the amount of calories consumed.

As used herein, the term “calorie” refers to a unit of energy. In one embodiment, a calorie is the approximate amount of energy required to raise the temperature of one gram of water by one degree Celsius at a pressure of one atmosphere. In one embodiment, a calorie refers to a kilogram calorie (symbol: Cal), defined in terms of kilograms rather than grams. A kilogram calorie is equal to 1000 small calories, i.e., 1 kilocalorie (symbol: kcal). Within the context of this disclosure, the term calorie normally refers to a kilocalorie often referred to as a “Calorie”. Calories are often used as a measurement to determine the amount of energy one should consume in order to properly fuel the body. In some embodiments, calories are used as a way to predict weight loss/gain. In one embodiment, calories are correlated with the volume of a segment.

In one embodiment of the methods disclosed herein, diet comprises food composition.

As used herein, the term “food composition” refers to the chemical makeup of substances. In some embodiments, food composition refers to the nutritional value of food. In one embodiment, food composition comprises the number of calories. In one embodiment, food composition comprises the amount of fat. In one embodiment, food composition comprises the amount of protein. In one embodiment, food composition comprises the amount of fiber. In one embodiment, food composition comprises the amount of sugar. In one embodiment, food composition comprises the amount of vitamins.

In one embodiment of the methods disclosed herein, exercise comprises time and heart rate.

As used herein, the term “time” refers to the measured or measurable period during which an action, process, or condition exists or continues. In one embodiment, time is measured in minutes. In one embodiment, time is measured in hours. In one embodiment, time is measured in days. In one embodiment, time is measured in months. In one embodiment, time is measured in years. In one embodiment, time is the measurement of how long the heart is exposed to a stimulus. In one embodiment, time is the amount of time the heart is monitored.

As used herein, the term “heart rate” refers to the speed of the heart in an amount of time. In one embodiment, the heart rate comprises heart contractions. In one embodiment, time and heart rate is measured in heartbeats per minute (hbm). In one embodiment, time and heart rate correlates to the volume of a segment. In one embodiment, the heart rate changes in response to a stimulus. In one embodiment, the stimulus is exercise. In one embodiment, the heart rate of a body is predicted to change.

In one embodiment of the methods disclosed herein, exercise comprises resistance and muscle group.

As used herein, the term “muscle” refers to soft tissue within in a body. Muscle cells contain protein filaments, e.g., actin and myosin, sliding past one another producing a contraction that changes both the length and the shape of the cell. In one embodiment, muscles function to produce force and motion. In one embodiment, muscles are primarily responsible for maintaining and changing posture, locomotion, as well as movement of internal organs, such as the contraction of the heart, and the movement of food through the digestive system via peristalsis. In one embodiment, the muscle is skeletal/striated. In one embodiment, the muscle is cardiac. In one embodiment, the muscle is smooth.

As used herein, the term “muscle group” refers to a classification of a band or bundle of fibrous tissue in the body having the ability to contract, produce movement, or maintain the position of parts of the body. In one embodiment, the muscle group is the pectoral. In one embodiment, the muscle group is the tricep. In one embodiment, the muscle group is the quadricep. In one embodiment, the muscle group is the calf. In one embodiment, the muscle group is the hamstring. In one embodiment, the muscle group is the forearm.

As used herein, the term “resistance” refers to exercise causing skeletal muscles to contract. In one embodiment, an external resistance, e.g., weights, causes contractions and those contractions lead to an increase in muscular mass, strength, endurance, and/or tone. In one embodiment, resistance is predicted to change the muscle group.

In one embodiment of the methods disclosed herein, predicting the change in volume comprises correlating the first stimulus with the plurality of second bodies. In one embodiment, the plurality of second bodies is a comparator.

In one embodiment, the methods disclosed herein comprise comparing a first body at a first age to a second body at the first age.

As used herein, the term “comparing” refers to determining the similarities and differences between two or more things. In one embodiment, comparing is between two bodies. In one embodiment, comparing is between two human bodies. In one embodiment, comparing comprises using a machine learning program. In one embodiment, comparing refers to juxtaposing two patches, such as patch A and patch B. For example, in one embodiment, comparing patch A and patch B means ordering the mean intensity of patch A and patch B. For example, patch A and patch B may be ordered where the mean intensity of patch A is greater than the mean intensity of patch B.

As used herein, the term “age” refers to the quantity of time of existence. In one embodiment, age is the quantity of time an organism has been alive. In one embodiment, age is measured in years. In one embodiment, age refers to the amount of time a body exists. In one embodiment, the body is human.

In one embodiment, the methods disclosed herein comprise identifying a volume for a segment of the second body at a second age. In one embodiment, the methods disclosed herein comprise identifying a volume for a segment of the third body at a third age. In one embodiment, the methods disclosed herein comprise identifying a volume for a segment of the fourth body at a fourth age. In one embodiment, the methods disclosed herein comprise identifying a volume for a segment of the fifth body at a fifth age.

In one embodiment, the methods disclosed herein comprise predicting a change in volume for a segment of the first body. In one embodiment, predicting a change in volume for a segment of the first body comprises using a machine learning program. In one embodiment, predicting a change in volume for a segment of the first body comprises using a cloud database platform. In one embodiment, predicting a change in volume for a segment of the first body comprises correlating a stimulus.

In one embodiment, the methods disclosed herein comprise a second stimulus. In one embodiment, the second stimulus is diet and exercise. In one embodiment, the second stimulus comprises caloric intake. In one embodiment, the second stimulus comprises food composition. In one embodiment, the second stimulus comprises time and heart rate. In one embodiment, the second stimulus comprises resistance and muscle group.

In one embodiment of the methods disclosed herein, predicting the change in volume comprises correlating the first stimulus with the second segment and correlating the second stimulus with the second segment. In one embodiment of the methods disclosed herein, predicting the change in volume comprises a machine learning program. In one embodiment of the methods disclosed herein, predicting the change in volume comprises a cloud database platform. In one embodiment of the methods disclosed herein, predicting the change in volume comprises a plurality of comparator bodies.

Within the context of this disclosure, the process steps or procedures can be executed by a human, operator, machine, and/or any combination thereof. For example, any combination of the following non-limiting exemplary steps could be executed by a human, operator, machine, and/or any combination thereof:
Converting DICOM to NIfTI (individual slabs)
Reassembling NIfTI slabs into full volume
Detecting/correcting cardiac artifacts
Estimating spinal cord
Estimating bone-joint landmarks
Detecting and segmenting individual vertebrae
Upsampling manual segmentations using random forests with geodesic features
Converting fat and water signal intensities to relative percentages
Estimating body mask
Computing “feature vector”
Detecting and segmenting male genitalia
Detecting and segmenting arms
Estimating boundary between the legs
Partitioning the body into anatomical regions
Segmenting the lungs and trachea
Segmenting the iliopsoas muscles
Segmenting the torso muscles (chest, back, abdomen and shoulders)
Detecting and segmenting the breasts
Segmenting the major leg muscles (upper and lower, left and right)
Segmenting the major leg bones (femur and tibia, left and right)
Segmenting pelvic bone and iliacus muscles (left and right)
Segmenting kidneys
Segmenting liver
Segmenting ribcage
Segmenting subcutaneous fat
Segmenting visceral fat
Segmenting internal thigh fat

Within the context of this disclosure, “Converting DICOM to NIfTI (individual slabs)” refers to assembling DICOM files together by series and converting into NIfTI volumes.

Within the context of this disclosure, “Reassembling NIfTI slabs into full volume” refers to merging all NIfTI series into a single whole-body volume. As well as automating and implementing fat-water swap detection/correction using only the subject's scan.

Within the context of this disclosure, “Detecting/correcting cardiac artifacts” refers to detecting motion-based cardiac artifacts and removing them to improve the quality of tissue segmentations.

Within the context of this disclosure, “Estimating spinal cord” refers to estimating a continuous curve following the contour of the spinal cord using random ferns. The location of the curve is posterior to and in between the spines of an individual vertebrae. This curve is used to exclude unwanted fat/muscle tissue when defining the abdominopelvic cavity for visceral fat segmentation.

Within the context of this disclosure, “Estimating bone-joint landmarks” refers to detecting the major bone joints (shoulders, hips, knees, ankles) using training data and machine learning techniques. The estimated locations form a coordinate system is unique to each subject and allows anatomically-specific partitions.

Within the context of this disclosure, “Detecting and segmenting individual vertebrae” refers to combining training datasets in a machine-learning framework to detect the centroids of the vertebrae (L5-T8). The results are used to exclude fatty tissue from the visceral fat segmentation.

Within the context of this disclosure, “Upsampling manual segmentations using random forests with geodesic features” refers to manually generated segmented organs/tissue, obtained in a downsampled space and upsampling them to full resolution using an interpolator that is guided by the signal intensities of the data.

Within the context of this disclosure, “Converting fat and water signal intensities to relative percentages” refers to overcoming the fat and water signal intensity discrepancies caused by inhomogeneities by converting them to relative percentages. They are also used to estimate the volume estimates related to fat and nonfat tissues.

Within the context of this disclosure, “Estimating body mask” refers to producing a binary mask including only tissue associated with the subject's body and separating the subject from air, the scanner table, etc.

Within the context of this disclosure, “Computing ‘feature vector’” refers to applying Principal components analysis (PCA) to each subject's body composition above the hips. This value is used to determine which subject's in the database are most similar to a new subject for atlas-based segmentation.

Within the context of this disclosure, “Detecting and segmenting male genitalia” refers to defining a search volume in a region based on the hip landmarks and assuming the genitals are the only area where fat is not located close to the body surface. The genitals are then excluded from the fat segmentation routines.

Within the context of this disclosure, “Detecting and segmenting arms” refers to transforming the coordinate system in order to identify all voxels associated with the arms and shoulders. The arms and some of the shoulder tissue are then excluded from quantitative analysis.

Within the context of this disclosure, “Estimating boundary between the legs” refers to splitting the whole body into left and right components with particular attention to separating the legs.

Within the context of this disclosure, “Partitioning the body into anatomical regions” refers to defining four major components: head, torso, left leg and right leg.

Within the context of this disclosure, “Segmenting the lungs and trachea” refers to using the lack of an MR signal to extract the lungs and trachea.

Within the context of this disclosure, “Segmenting the iliopsoas muscles” refers to atlas-based segmentation and refinement procedures applied to the iliopsoas muscles and manually generated training data.

Within the context of this disclosure, “Segmenting the torso muscles (chest, back, abdomen, and shoulders)” refers to atlas-based segmentation and refinement procedures applied to the torso muscles and manually generating training data.

Within the context of this disclosure, “Detecting and segmenting the breasts” refers to removing the non-fat breast tissue from fat segmentations using the mask of breast tissue from the torso segmentation. The mask of breast tissue from the torso segmentation is derived from manual or atlas-based segmentation techniques. Atlas-based segmentation and refinement procedures are then applied to produce a final segmentation.

Within the context of this disclosure, “Segmenting the major leg muscles (upper and lower, left and right)” refers to generating an initial mask of the left and right legs using only the subject's data. Atlas-based segmentation and refinement are then applied to produce a final segmentation.

Within the context of this disclosure, “Segmenting the major leg bones (femur and tibia, left and right)” refers to initially segmenting the femur and tibia based on the initial leg segmentation and the detected landmarks of the bone joints. The method is based on region growing and geodesic distances. Atlas-based segmentation and refinement procedures are then applied to produce a final segmentation.

Within the context of this disclosure, “Segmenting pelvic bone and iliacus muscles (left and right)” refers to atlas-based segmentation and refinement procedures applied to the pelvic bone and iliacus muscles and manually generated training data.

Within the context of this disclosure, “Segmenting kidneys” refers to atlas-based segmentation and refinement procedures applied to the kidneys and manually generated training data.

Within the context of this disclosure, “Segmenting liver” refers to atlas-based segmentation and refinement procedures applied to the liver and manually generated training data.

Within the context of this disclosure, “Segmenting ribcage” refers to estimating the position of a thin surface containing the ribs and using a rib shape model and registration on the fat percentages.

Within the context of this disclosure, “Segmenting subcutaneous fat” refers to first defining the body cavity and then estimating all fat tissue between the body cavity and boundary of the body.

Within the context of this disclosure, “Segmenting visceral fat” refers to defining the abdominopelvic cavity and eliminating all other tissues and non-relevant organs. Then, estimating all fat tissue within the abdominopelvic cavity.

Within the context of this disclosure, “Segmenting internal thigh fat” refers to using the leg bone and subcutaneous fat segmentations for segmenting the remaining fat and muscle tissue in the upper legs. The midpoint between the hips and knees is estimated and a fixed region of muscle tissue is defined.

In one embodiment, 20 subjects are matched to a current body to provide atlases for each muscle group. The torso/iliopsoas atlases are generated by a human operator. Then, leg atlases are generated automatically. Atlas-based registration construct a probability mask. Refinement for torso-muscle segmentation are made using graph cuts (continuous max flow). Refinement of leg-muscle segmentation are made using conditional random field (dense CRF). There is no refinement of the iliopsoas segmentation, only thresholding.

In one embodiment, data from single MRI is used. A human operator estimates the body cavity as well estimating the subcutaneous fat by excluding the body cavity. Refinements of both segmentations are done automatically by conditional random field (dense CRF). Unions and intersections of anatomy are used to estimate the abdominal cavity. Then the body cavity, pelvic mask, lungs, spine, upper legs, torso muscle, and iliopsoas muscle are estimated. Only the visceral fat is estimated in the abdominal cavity.

In one embodiment, thigh muscles are isolated. A human operator segments the subcutaneous fat from the thigh. A machine learning program automatically removes the skeletal structure. The fat fraction is calculated from a Dixon MRI. Mapping T2 and fc-T2 is from additional sequencing.

Although the disclosed invention has been described with reference to various exemplary embodiments, it is to be understood that these embodiments are merely illustrative of the principles and applications of the present invention. Those having skill in the art would recognize that various modifications to the exemplary embodiments may be made, without departing from the scope of the invention.

Moreover, it should be understood that various features and/or characteristics of differing embodiments herein may be combined with one another. It is therefore to be understood that numerous modifications may be made to the illustrative embodiments and that other arrangements may be devised without departing from the scope of the invention.

Furthermore, other embodiments of the invention will be apparent to those skilled in the art from consideration of the specification and practice of the invention disclosed herein. It is intended that the specification and examples be considered as exemplary only, with a scope and spirit being indicated by the claims.

Finally, it is noted that, as used in this specification and the appended claims, the singular forms “a,” “an,” and “the,” include plural referents unless expressly and unequivocally limited to one referent, and vice versa. As used herein, the term “include” or “comprising” and its grammatical variants are intended to be non-limiting, such that recitation of an item or items is not to the exclusion of other like items that can be substituted or added to the recited item(s).

What is claimed is:

1. A method of predicting compositional change in a body comprising:
Collecting a plurality of two-dimensional images for a first body;
Collecting a plurality of two-dimensional images for a second body;
Processing the plurality of two-dimensional body images for the first body into a first three-dimensional representation of the first body;
Processing the plurality of two-dimensional body images for the second body into a second three-dimensional representation of the second body;
Assigning landmarks to points within the first three-dimensional representation of the first body;
Assigning landmarks to points within the second three-dimensional representation of the second body;
Registering the first three-dimensional representation of the first body;
Registering the second three-dimensional representation of the second body;
Segmenting the first three-dimensional representation of the first body into a first segment;
Segmenting the second three-dimensional representation of the second body into a second segment;
Calculating a volume of the first segment;
Calculating a volume of the second segment;
Identifying a first stimulus; and
Predicting a change in the volume of the first segment.

2. The method of claim 1, wherein predicting the change in volume comprises correlating the first stimulus with the second segment.

3. The method of claim 1, comprising a plurality of second bodies.

4. The method of claim 1, wherein the first stimulus is chosen from diet and exercise.

5. The method of claim 4, wherein the diet comprises caloric intake.

6. The method of claim 4, wherein the diet comprises food composition.

7. The method of claim 4, wherein the exercise comprises time and heart rate.

8. The method of claim 4, wherein the exercise comprises resistance and muscle group.

9. The method of claim 3, wherein predicting the change in volume comprises correlating the first stimulus with the plurality of second bodies.

10. The method of claim 1, comprising comparing a first body at a first age to a second body at the first age.

11. The method of claim 10, comprising identifying a volume for a segment of the second body at a second age.

12. The method of claim 11, comprising predicting a change in volume for a segment of the first body.

13. The method of claim 1, comprising a second stimulus.

14. The method of claim 13, wherein predicting the change in volume comprises correlating the first stimulus with the second segment and correlating the second stimulus with the second segment.
